(19) United States
(12) Patent Application Publication
(10) Pub. No.: US 2021/0045305 A1
(43) Pub. Date: Feb. 18, 2021

(54) AUTOFLOCCULATION OF ALGAL BIOMASS

(71) Applicant: ExxonMobil Research and Engineering Company, Annandale, NJ (US)

(72) Inventors: Everett J. O'Neal, Asbury, NJ (US); Mark A. Deimund, Jersey City, NJ (US)

(21) Appl. No.: 16/891,136

(22) Filed: Jun. 3, 2020

Related U.S. Application Data
(60) Provisional application No. 62/888,032, filed on Aug. 16, 2019.

(51) Int. Cl. A01G 33/00; C12N 1/02; C12M 1/34

(52) U.S. Cl. CPC A01G 33/00; C12M 41/32; C12M 41/26; C12N 1/02

(57) ABSTRACT

Methods for autoflocculation of algae cells in an algae water slurry to facilitate harvesting. Algae cells are cultivated in the algae water slurry to produce an algal biomass and thereafter autoflocculated to allow for an enhanced dewatering harvesting process. Autoflocculation provides a cost-effective and efficient harvesting method compared to traditional dewatering processes.

### CROSS-REFERENCE TO RELATED APPLICATIONS

This application claims the benefit of U.S. Provisional Application No. 62/888,032 filed Aug. 16, 2019, which is herein incorporated by reference in its entirety.

### BACKGROUND OF THE INVENTION

Concerns about climate change, carbon dioxide (CO₂) emissions, and depleting mineral oil and gas resources have led to widespread interest in the production of biofuels from algae, including microalgae. As compared to other plant-based feedstocks, algae have higher CO₂ fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO₂ combines with solar energy, other nutrients, and cellular biochemical processes to output gaseous oxygen and to synthesize carbohydrates and other compounds critical to the life of the algae.

To produce algal biomass, algae is generally grown in a water slurry comprising water and nutrients. The algae may be cultivated in indoor or outdoor environments, and in closed or open cultivation systems. Closed cultivation systems include photobioreactors, which utilize natural or artificial light to grow algae in an environment that is generally isolated from the external atmosphere. Such photobioreactors may be in a variety of shaped configurations, but are typically tubular or flat paneled. Open cultivation systems include natural and artificial ponds that utilize sunlight to facilitate photosynthesis. Artificial ponds are generally more preferred for industrial, scaled-up cultivation and are often shaped in circular or raceway-shaped configurations.

Various processing methods exist for harvesting cultivated algal biomass to extract lipids therefrom for the production of fuel and other oil-based products. Moreover, harvesting cultivated algal biomass can be used to produce non-fuel or non-oil-based products, including nutraceuticals, pharmaceuticals, cosmetics, chemicals (e.g., paints, dyes, and colorants), fertilizer and animal feed, and the like. Such methods include the addition of chemicals or the use of mechanical equipment to physically separate algae from the remaining components of a water slurry. Separation of algal biomass has proven to be a dramatic drain on production costs because harvest-ready algae is typically in low concentrations in a water slurry (e.g., about 1 gram per liter), has low sedimentation velocity, and has a colloidal nature that maintains it in suspension, among other complications. As such, algae does not itself easily settle out of a water slurry and, large volumes of liquid must be processed to concentrate algal biomass.

Because the processing of algal biomass produces valuable commodities, including sustainable biofuels, cost-effective harvesting methods that overcome some or all of the complications traditionally associated with harvesting are desirable. Moreover, it is further desirable that such harvesting methods minimize energy usage and minimize chemical exposure to decrease environmental impact and decrease associated production costs.

### SUMMARY OF THE INVENTION

The present disclosure is related to autoflocculation of algal biomass to facilitate harvesting, and more particularly, to autoflocculation of algal biomass based on controlling the isoelectric point of algae.

In some embodiments, a method, as disclosed herein, includes a cultivation vessel containing an algae water slurry comprising algae cells, water, and algae nutrient media. The algae water slurry is cultivated for a predetermined time within the cultivation vessel and thereafter the algae cells are autoflocculated by driving the cultivated algae water slurry toward an isoelectric point of the algae cells.

In some embodiments, a method, as disclosed herein, includes a cultivation vessel containing an algae water slurry comprising algae cells, water, and algae nutrient media. The algae water slurry is cultivated for a predetermined time within the cultivation vessel and thereafter the algae cells are autoflocculated by modifying a surface charge of the algae cells by controlling solution conditions of the cultivated algae water slurry.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a schematic illustration of an autoflocculation method based on a high-salinity shock approach, according to one or more embodiments of the present disclosure.

FIG. 2 is a schematic illustration of an autoflocculation method based on a change in pH approach, according to one or more embodiments of the present disclosure.

### DETAILED DESCRIPTION

The present disclosure is related to autoflocculation of algal biomass to facilitate harvesting, and more particularly, to autoflocculation of algal biomass based on controlling the isoelectric point of algae.

Biofuel production from cultivated algae slurries offers sustainable energy solutions to reduce reliance on fossil fuels and reduce greenhouse gas emissions. To accomplish substantial economic, environmental, and societal impact, algae must be cultivated in large-scale systems. Such large-scale cultivation systems allow algae-derived fuels to become more cost-effective and more widely available to the public. However, harvesting algal biomass is traditionally a major bottleneck to large-scale, industrial sized processing in terms of production costs, time, and environmental impact, requiring algal biomass to be effectively separated, or “dewatered,” from the remaining components of large-volume slurries. As such, traditional harvesting methods can render algae-based biofuels less attractive.

Traditional harvesting techniques may employ one or more biological, chemical, mechanical, and/or electrical operations, such as centrifugation, filtration, sedimentation, flotation, and the like. Flocculation of algae cells in a slurry can facilitate or ease harvesting by such methods by reducing the amount of slurry liquid that must be processed to separate the algal biomass therefrom. The embodiments described herein provide methods that promote, stimulate, and otherwise encourage autoflocculation of algae to facilitate harvesting algal biomass with decreased associated costs and energy consumption compared to traditional methods. Such autoflocculation methods are effective, reliable, and manageable using minimal capital and operational energy when applied to large-scale cultivation systems. Moreover, the autoflocculation methods described herein do not employ inorganic or organic chemical flocculants, further decreasing costs and environmental impact.

As used herein, the term “algae slurry” or “algae water slurry,” and grammatical variants thereof, refers to a flowable, liquid comprising at least water, algae cells, and algae nutrient media (e.g., phosphorous, nitrogen, and optionally additional elemental nutrients).

As used herein, the term “autoflocculation,” and grammatical variants thereof, refers to spontaneous aggregation (accumulation) of algae cells. Autoflocculation permits the algae cells settle out of their colloid suspension within an algae slurry.

As used herein, the term “cultivation vessel,” and grammatical variants thereof, refers to any of an open or closed cultivation system used for the growth of algal biomass, including photobioreactors, natural ponds, artificial ponds (e.g., raceway ponds), and the like.

Algae cells comprise charged surface proteins and other charged compounds that generally prevent their natural settling within a slurry liquid. The zeta potential of algae cells is a measure of these surface changes within an algae slurry, defined as the potential difference between the algae cells and the slurry liquid. Typically, the zeta potential of algae cells is negative due to negatively charged proteins and other compounds existing on the surface of the cells. These charged surface compounds operate to maintain cultivated algal biomass in a colloidal suspension within a slurry because the charges repel one another. The typical zeta potential of algae cells is generally in the range of about −15 millivolts (mV) to about −40 mV, which is sufficient to maintain the cells in the colloid suspension. By manipulating an algae slurry to offset these charges and otherwise modifying the surface charges by controlling the solution conditions, algal biomass can autoflocculate and be easily separated from the remaining components of the slurry to facilitate harvesting. More particularly, an algae slurry is manipulated to achieve the isoelectric point of the algae cells therein, which is the pH of the slurry at which the zeta potential (or net charge) of the algae cells is at or near zero (0). As used herein, a zeta potential “at or near zero (0)” is defined as in the range of about +5 mV to about −5 mV, encompassing any value and subset therebetween. At or near the isoelectic point (i.e., at a neutralized or near-neutralized zeta potential charge), the components of the slurry are no longer stable, thus allowing the algae cells of the cultivated algal biomass to precipitate and autoflocculate from the slurry medium.

The methods described herein manipulate algae slurries to achieve the isoelectric point by “shocking” the algae cells therein using dramatic changes in salinity and/or pH to reduce or eliminate the otherwise charged nature of the algae cells. That is, the algae cells are autoflocculated by controlling solution conditions and thereby driving a cultivated slurry toward the isoelectric point. This can be accomplished through the addition of nutrients or other chemicals that alter the salinity and/or pH of the slurry, such as carbon dioxide (CO₂), salt, fresh water, salt water, and the like, and any combination thereof to cause autoflocculation. The required change in salinity and/or pH to drive autoflocculation may be determined based on the zeta potential of the particular species of algae grown. The zeta potential of algae cells can be determined through laboratory methods, such as by use of a zeta potential analyzer. Based on the known zeta potential, the particular pH and/or salinity change of an algae slurry can be determined (e.g., through titration experimentation) that will drive the slurry toward the isoelectric point and cause autoflocculation.

According to one or more embodiments, an algae culture “seed stock” may be initially prepared. Algal sources for the preparing the seed stock include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

An algae water slurry may be prepared using the seed stock, water, and algae nutrient media. The water for use in preparing the algae slurry may be from any water source including, but not limited to, fresh water, brackish water, seawater, wastewater (treated or untreated), synthetic seawater, and any combination thereof. The wastewater may derive, for example, from previously cultivated algae slurries after separation and removal of the algae components. The synthetic seawater may, for example, be prepared by dissolving salts into fresh water.

The algae nutrient media for use in forming an algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

In accordance with the methods described herein, cultivation of the algae water slurry may be performed in closed or open cultivation systems. In some instances, for example, the type of cultivation system may be selected based on the particular method to be used for achieving autoflocculation. For example, if the salinity of a slurry is increased by natural evaporation, an open outdoor cultivation system may be preferred. Alternatively, if the pH of a slurry is altered by sparging carbon dioxide into a slurry, the open or closed nature of a cultivations system may be less important. Regardless of the particular cultivation system selected, autoflocculation of algae cells from a slurry allows for simplified dewatering and separation from other slurry components for harvesting.

According to one or more embodiments of the present disclosure, an algae slurry is prepared or otherwise mixed in a cultivation vessel (closed or open system) and cultivated for a predetermined period of time to grow algal biomass. In some embodiments, the predetermined period of time for cultivation may be between about 12 hours to about 5 weeks, encompassing any value and subset therebetween, such as about 12 hours to about 3 weeks. Thereafter, the salinity and/or the pH of the cultivated algal biomass slurry may be treated or otherwise naturally altered to cause the algae cells to autoflocculate.

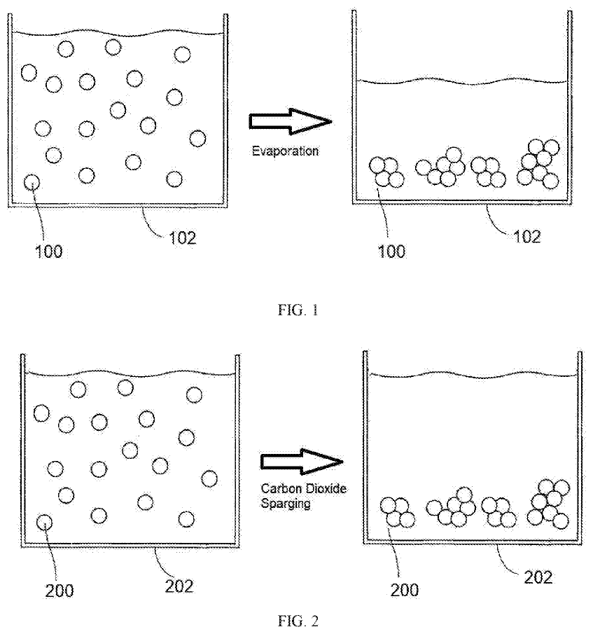

Referring now to FIG. 1, illustrated is an embodiment of an autoflocculation method of the present disclosure based on a high-salinity shock approach. In the illustrated embodiment, an algae slurry is cultivated using algae cells 100, seawater, and algae nutrient media in an open, outdoor pond 102. The algae slurry has an initial salinity, such as about 7% by weight, and during cultivation of the algae cells 100 to form algal biomass, the cells 100 remain in suspension in the slurry. As the end of the predetermined cultivation period nears (e.g., one to two days prior to the end of the period), natural evaporation may be used (relied upon) to drive the salinity of the slurry higher, such as to about 10% by weight. Accordingly, the ionic strength of the growth medium increases, which drives the slurry toward the isoelectric point to cause the algae cells 100 to autoflocculate.

After autoflocculation, various means may be used to recover the algae cells 100, such as by draining or otherwise decanting the liquid slurry components from the top of the pond 102. Any non-autoflocculated algae cells 102 may be skimmed (e.g., using a weir) from the slurry. The recovered higher salinity liquid (e.g., the 10% by weight salinity liquid) can also be recycled and used to cultivate a new seed stock, thereby reducing the amount of total seawater needed for the subsequent cultivation.

Referring now to FIG. 2, illustrated is another embodiment of an autoflocculation method of the present disclosure based on a change in pH approach. In the illustrated embodiment, an algae slurry is cultivated using algae cells 200, seawater, and algae nutrient media in an open, outdoor pond 202. In other embodiments, the pond 200 may comprise a closed photobioreactor, without departing from the scope of the disclosure. The algae slurry has an initial salinity and pH with is optimum for the growth of the particular strain of algae cells. During cultivation of the algae cells 200 to form algal biomass, the cells remain in suspension in the slurry. At the end of the predetermined cultivation period, the pH of the slurry may be reduced by sparging CO₂ (which is typically used to provide a carbon source for algae growth) into the slurry. The CO₂ reduces the pH to as low or lower than about 6.0, and thus modifies the surface charge of the algae cells 200, which drives the slurry toward the isoelectric point to cause the algae cells 200 to autoflocculate. The autoflocculated algae cells 200 (and any non-autoflocculated algae cells) may be recovered as described with reference to FIG. 1, or by other means known by those of skill in the art.

### Embodiments Listing

The present disclosure provides, among others, the following embodiments, each of which may be considered as optionally including any alternate embodiments.

Clause 1. A method comprising: containing an algae water slurry in a cultivation vessel, the algae water slurry comprising algae cells, water, and algae nutrient media; cultivating the algae water slurry for a predetermined period of time; and autoflocculating the algae cells by driving the cultivated algae water slurry toward an isoelectric point of the algae cells.

Clause 2. The method of Clause 1, further comprising determining a zeta potential of the algae cells prior to autoflocculating.

Clause 3. The method of any of the preceding Clauses, wherein autoflocculating the algae cells comprises altering a pH value of the algae water slurry.

Clause 4. The method of Clause 3, wherein altering the pH value of the algae water slurry comprises reducing the pH.

Clause 5. The method of Clause 3 or 4, further comprising sparging carbon dioxide into the algae water slurry to alter the pH.

Clause 6. The method of Clause 1 or 2, wherein autoflocculating the algae cells comprises altering a salinity of the algae water slurry.

Clause 7. The method of Clause 6, wherein altering the salinity of the algae water slurry comprises increasing the salinity.

Clause 8. The method of Clause 3 or 7, further comprising evaporating a portion of the water in the algae water slurry to alter the salinity.

Clause 9. The method of any of the preceding Clauses, further comprising separating the autoflocculated algae cells from the water and the algae nutrient media.

Clause 10. The method of any of the preceding Clauses, wherein the predetermined period of time is in the range of about 12 hours to about 5 weeks.

Clause 11. The method of any of the preceding Clauses, wherein the algae cells are one or more of unicellular and multicellular.

Clause 12. The method of any of the preceding Clauses, wherein the water is selected from the group consisting of fresh water, brackish water, seawater, wastewater (treated or untreated), synthetic seawater, and any combination thereof.

Clause 13. A method comprising: containing an algae water slurry in a cultivation vessel, the algae water slurry comprising algae cells, water, and algae nutrient media; cultivating the algae water slurry for a predetermined period of time; and modifying a surface charge of the algae cells by controlling solution conditions of the algae water slurry and thereby autoflocculating the algae cells.

Clause 14. The method of Clause 13, wherein controlling the solution conditions of the algae water slurry comprises altering a pH value of the algae water slurry.

Clause 15. The method of Clause 14, wherein altering the pH value of the algae water slurry comprises reducing the pH.

Clause 16. The method of Clause 14 or 15, further comprising sparging carbon dioxide into the algae water slurry to alter the pH.

Clause 17. The method of Clause 13, wherein controlling the solution conditions of the algae water slurry comprises altering a salinity of the algae water slurry.

Clause 18. The method of Clause 17, wherein altering the salinity of the algae water slurry comprises increasing the salinity.

Clause 19. The method of Clause 17 or 18, further comprising evaporating a portion of the water in the algae water slurry to alter the salinity.

Clause 20. The method of Clause 13-19, further comprising separating the autoflocculated algae cells from the water and the algae nutrient media.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C

The invention claimed is:

1. A method comprising:
containing an algae water slurry in a cultivation vessel, the algae water slurry comprising algae cells, water, and algae nutrient media;
cultivating the algae water slurry for a predetermined period of time; and
autoflocculating the algae cells by driving the cultivated algae water slurry toward an isoelectric point of the algae cells.

2. The method of claim 1, further comprising determining a zeta potential of the algae cells prior to autoflocculating.

3. The method of claim 1, wherein autoflocculating the algae cells comprises altering a pH value of the algae water slurry.

4. The method of claim 3, wherein altering the pH value of the algae water slurry comprises reducing the pH.

5. The method of claim 3, further comprising sparging carbon dioxide into the algae water slurry to alter the pH.

6. The method of claim 1, wherein autoflocculating the algae cells comprises altering a salinity of the algae water slurry.

7. The method of claim 6, wherein altering the salinity of the algae water slurry comprises increasing the salinity.

8. The method of claim 6, further comprising evaporating a portion of the water in the algae water slurry to alter the salinity.

9. The method of claim 1, further comprising separating the autoflocculated algae cells from the water and the algae nutrient media.

10. The method of claim 1, wherein the predetermined period of time is in the range of about 12 hours to about 5 weeks.

11. The method of claim 1, wherein the algae cells are one or more of unicellular and multicellular.

12. The method of claim 1, wherein the water is selected from the group consisting of fresh water, brackish water, seawater, wastewater (treated or untreated), synthetic seawater, and any combination thereof.

13. A method comprising:
containing an algae water slurry in a cultivation vessel, the algae water slurry comprising algae cells, water, and algae nutrient media;
cultivating the algae water slurry for a predetermined period of time; and
modifying a surface charge of the algae cells by controlling solution conditions of the algae water slurry and thereby autoflocculating the algae cells.

14. The method of claim 13, wherein controlling the solution conditions of the algae water slurry comprises altering a pH value of the algae water slurry.

15. The method of claim 14, wherein altering the pH value of the algae water slurry comprises reducing the pH.

16. The method of claim 14, further comprising sparging carbon dioxide into the algae water slurry to alter the pH.

17. The method of claim 13, wherein controlling the solution conditions of the algae water slurry comprises altering a salinity of the algae water slurry.

18. The method of claim 17, wherein altering the salinity of the algae water slurry comprises increasing the salinity.

19. The method of claim 17, further comprising evaporating a portion of the water in the algae water slurry to alter the salinity.

20. The method of claim 13, further comprising separating the autoflocculated algae cells from the water and the algae nutrient media.
